(19) United States
(12) Patent Application Publication
(10) Pub. No.: US 2026/0112257 A1
(43) Pub. Date: Apr. 23, 2026

(54) SYSTEMS AND METHODS FOR ENHANCING COMMUNICATION ABILITIES OF DATA PROCESSING SYSTEMS USING OUT-OF-BAND COMPONENTS

(71) Applicant: Dell Products L.P., Round Rock, TX (US)

(72) Inventors: RICHARD M. TONRY, Georgetown, TX (US); ABEYE TESHOME, Austin, TX (US); BASSEM EL-AZZAMI, Austin, TX (US); MOHIT ARORA, Frisco, TX (US); VINODKUMAR VASUDEV OTTAR, McKinney, TX (US); ADOLFO SANDOR MONTERO, Pflugerville, TX (US); LUIS ANTONIO VALENCIA REYES, Waxahachie, TX (US); RAJARAVI CHANDRA KOLLARAPU, Allen, TX (US)

(21) Appl. No.: 18/920,120

(22) Filed: Oct. 18, 2024

(51) Int. Cl. G08B 21/02; G08B 17/00; G16H 10/60; H04L 67/1061

(52) U.S. Cl. CPC G08B 21/02; G08B 17/00; G16H 10/60; H04L 67/1061

(57) ABSTRACT

Methods and systems for managing operation of a data processing system are disclosed. To manage operation of the data processing system, an occurrence of an emergency alert event may be identified by a management controller of the data processing system. Based on the identified emergency alert event, the management controller may initiate an emergency alert process and perform the emergency alert process in cooperation with a management system and emergency services system in order to update a state of the data processing system. By doing so, the likelihood of data processing system being able to communicate with remote entities that are required to cooperatively provide the computer implemented services may be increased.

### FIELD

Embodiments disclosed herein relate generally to managing a data processing system. More particularly, embodiments disclosed herein relate to systems and methods for managing operations of data processing systems.

### BACKGROUND

Computing devices may provide computer-implemented services. The computer-implemented services may be used by users of the computing devices and/or devices operably connected to the computing devices. The computer-implemented services may be performed with hardware components such as processors, memory modules, storage devices, and communication devices. The operation of these components and the components of other devices may impact the performance of the computer-implemented services.

### BRIEF DESCRIPTION OF THE DRAWINGS

Embodiments disclosed herein are illustrated by way of example and not limitation in the figures of the accompanying drawings in which like references indicate similar elements.

FIG. 1A shows a block diagram illustrating a system in accordance with an embodiment.

FIG. 1B shows a block diagram illustrating a data processing system in accordance with an embodiment.

FIG. 2A shows an interaction diagram in accordance with an embodiment.

FIG. 3 shows a flow diagram illustrating a method of managing operation of a data processing system in accordance with an embodiment.

FIG. 4 shows a block diagram illustrating a data processing system in accordance with an embodiment.

### DETAILED DESCRIPTION

Various embodiments will be described with reference to details discussed below, and the accompanying drawings will illustrate the various embodiments. The following description and drawings are illustrative and are not to be construed as limiting. Numerous specific details are described to provide a thorough understanding of various embodiments. However, in certain instances, well-known or conventional details are not described in order to provide a concise discussion of embodiments disclosed herein.

Reference in the specification to “one embodiment” or “an embodiment” means that a particular feature, structure, or characteristic described in conjunction with the embodiment can be included in at least one embodiment. The appearances of the phrases “in one embodiment” and “an embodiment” in various places in the specification do not necessarily all refer to the same embodiment.

References to an “operable connection” or “operably connected” means that a particular device is able to communicate with one or more other devices. The devices themselves may be directly connected to one another or may be indirectly connected to one another through any number of intermediary devices, such as in a network topology.

In general, embodiments disclosed herein relate to methods and systems for managing (operation of) a data processing system. The data processing system may provide computer implemented services to users of the data processing system. The computer implemented services may include any quantity and type of such services. Some types of the computer implemented services may require an ability for the data processing system to communicate with other remote entities (e.g., other data processing systems, external devices, etc.). For example, To cooperatively provide computer implemented services, data processing system 100 may require ability to communicate with emergency service system 106. However, data processing system 100 may lack the capability (e.g., due to hardware architecture) to communicate (e.g., exchange data) with emergency service system 106. For example, hardware resources of data processing system 100 may become compromised (e.g., by a malicious entity) and thereby, limit ability of data processing system 100 to communicate (e.g., provide alert notifications, messages, etc.) with emergency service system 106.

In general, embodiments disclosed herein may provide methods, systems, and/or devices for managing operation of data processing system to establish communication capabilities with remote entities based on occurrence of an emergency alert event (e.g., any type of incident impacting an end user of the data processing system that requires emergency services to be provided). To do so, the data processing system may include out-of-band components that may communicate with one another without traversing in-band communication channels and without utilizing in-band components.

For example, the out-of-band components may monitor operation of in-band components (e.g., hardware resources and/or software resources hosted by the hardware resources) to identify indictors associated with emergency alert events (e.g., alert triggers). The out-of-band components may utilize the alert triggers to identify alert policies (e.g., guidelines, instructions, etc. to manage the identified emergency alert event) and initiate performance of an emergency alert process (e.g., based on the identified alert policy). During initiation of the emergency alert process, the out-of-band components may communicate the emergency alert (e.g., via an out-of-band communication channel) with a management system (e.g., a management system connected to telecommunication network), and upon receipt of the emergency alert, the management system may initiate communication (via the telecommunication network) with the emergency service system (e.g., system providing the emergency services) in order to establish a bridge for communications between the data processing system and the emergency service system. By doing so, the likelihood of the data processing system being able to communicate with remote entities that are required to cooperatively provide the computer implemented services may improve despite hardware architecture limitations of the data processing system impacting communications with the remote entities.

By doing so, embodiments disclosed herein may provide a system for managing (operation of) a data processing system based identified occurrences of emergency alert events. To do so, the data processing system may include out-of-band components such as a management controller. The management controller may, for example, identify various indications of activity (e.g., of the components of the data processing system) that may impact the safety and/or health of the data processing system and/or user of the data processing system. Based on the identified emergency alert event, the management controller may identify applicable policies and/or may perform an action set in cooperation with other entities order to update operation of the data processing system. Once updated, the data processing system may be more likely to provide desired computer implemented services (e.g., secure, uninterrupted, and/or otherwise policy compliant computer implemented services).

In an embodiment, a method for managing operation of a data processing system is provided. The method may include: identifying, by a management controller of the data processing system, an occurrence of an emergency alert event for the data processing system; based on the occurrence of the emergency alert event: initiating, by the management controller and in cooperation with a management system for the data processing system, an emergency alert process, the emergency alert process being cooperatively performed by an emergency services system and at least hardware resources of the data processing system to update a state of the data processing system; and providing, by the data processing system, computer implemented services based on the updated state of the data processing system.

Identifying the occurrence of the emergency alert event may include: monitoring, by the management controller, operation of the hardware resources, the operation of the hardware resources being indicative of occurrences of the emergency alert event.

Monitoring the operation of the hardware resources may include: programming, by the management controller, an agent hosted by the hardware resources.

The agent may be programmed to screen for at least one selected from a group consisting of: invocation, by a user of the data processing system, a hotkey combination; providing, by an auxiliary device and to the hardware resources, information indicative of the occurrence of the emergency alert event; and information, by a remote device and the hardware resources, information indicative of the occurrence of the emergency alert event.

The auxiliary device may be one selected from a group consisting of: a health state monitor; and a personal computing device.

The health state monitor may be one selected from a group consisting of: a heart rate monitor; and a glucose monitor.

The personal computing device may be a body worn computing device that includes at least one sensor for monitoring a user of the data processing system.

The remote device may be a safety system tasked with assisting in managing safety of users of the data processing system.

The safety system may be a fire management system.

The data processing system may include a network module adapted to separately advertise network endpoints for the management controller and hardware resources of the data processing system, the network endpoints being usable by the management system and the emergency services system to address communications to the hardware resources using an in-band communication channel and the management controller using an out-of-band communication channel.

The management controller and the network module may be on separate power domains from the hardware resources so that the management controller and the network module may be operable while the hardware resources are inoperable.

The out-of-band communication channel may run through the network module, and the in-band communication channel that services the hardware resources may also run through the network module.

The network module may host a transmission control protocol/internet protocol (TCP/IP) stack to facilitate network communications via the out-of-band communication channel.

In an embodiment, a non-transitory media is provided that may include instructions that when executed by a processor cause the computer-implemented method to be performed.

In an embodiment, a data processing system is provided that may include the non-transitory media and a processor, and may perform the computer-implemented method when the computer instructions are executed by the processor.

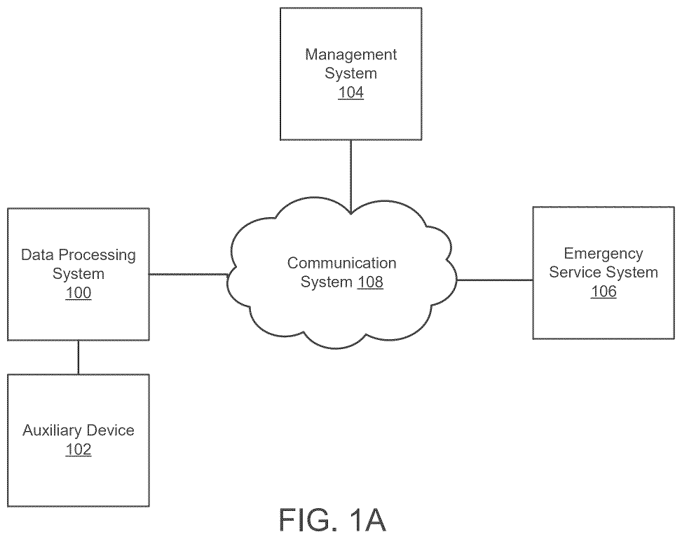

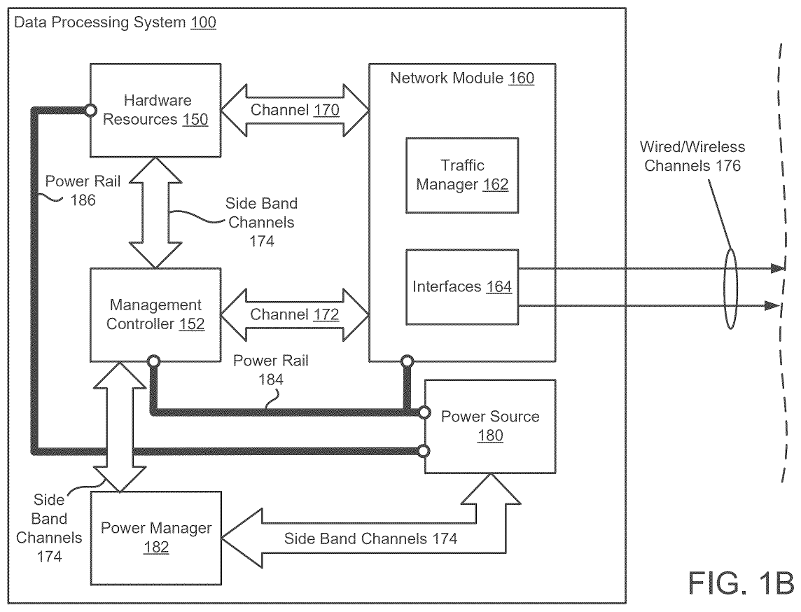

Turning to FIG. 1A, a block diagram illustrating a system in accordance with an embodiment is shown. The system shown in FIG. 1A may provide for management of data processing systems that may provide, at least in part, computer-implemented services. The system may include data processing system 100 (e.g., computing devices) that may include any number of hardware components (e.g., processors, memory modules, storage devices, communications devices). The hardware components may support execution of any number and types of applications (e.g., software components). Changes in available functionalities of the hardware and/or software components may provide for various types of different computer-implemented services to be provided over time. Refer to FIG. 1B for additional details regarding data processing system 100.

The computer implemented services may be provided cooperatively with emergency service system 106. Emergency service system 106 may include any type of systems (e.g., data processing system, computing device, cloud system, server, etc.) capable of providing emergency related services. The emergency services may include, for example, medical services, fire management, safety services, and/or any other type of emergency services to be provided to a user of a data processing system (e.g., data processing system 100).

To cooperatively provide computer implemented services, data processing system 100 may require ability to communicate with emergency service system 106. However, data processing system 100 may lack the capability (e.g., due to hardware architecture) to communicate (e.g., exchange data) with emergency service system 106. For example, hardware resources of data processing system 100 may become compromised (e.g., by a malicious entity) and thereby, limit ability of data processing system 100 to communicate (e.g., provide alert notifications, messages, etc.) with emergency service system 106.

In general, embodiments disclosed herein may provide methods, systems, and/or devices for managing operation of data processing system to establish communication capabilities with remote entities based on occurrence of an emergency alert event (e.g., any type of incident impacting an end user of the data processing system that requires emergency services to be provided). To do so, the data processing system may include out-of-band components that may communicate with one another without traversing in-band communication channels and without utilizing in-band components.

For example, the out-of-band components may monitor operation of in-band components (e.g., hardware resources and/or software resources hosted by the hardware resources) to identify indictors associated with emergency alert events (e.g., alert triggers). The out-of-band components may utilize the alert triggers to identify alert policies (e.g., guidelines, instructions, etc. to manage the identified emergency alert event) and initiate performance of an emergency alert process (e.g., based on the identified alert policy). During initiation of the emergency alert process, the out-of-band components may communicate the emergency alert (e.g., via an out-of-band communication channel) with a management system (e.g., a management system connected to telecommunication network), and upon receipt of the emergency alert, the management system may initiate communication (via the telecommunication network) with the emergency service system (e.g., system providing the emergency services) in order to establish a bridge for communications between the data processing system and the emergency service system. By doing so, the likelihood of the data processing system being able to communicate with remote entities that are required to cooperatively provide the computer implemented services may improve despite hardware architecture limitations of the data processing system impacting communications with the remote entities.

To perform the above-mentioned functionality, the system of FIG. 1A may include data processing system 100, auxiliary device 102, management system 104, and/or emergency service system 106. Data processing system 100, auxiliary device 102, management system 104, and/or emergency service system 106, and/or any other type of devices not shown in FIG. 1A may perform all, or a portion of the computer implemented services independently and/or cooperatively. Each of these components is discussed below.

Data processing system 100 may include any number and/or type of data processing systems (e.g., 100-100N). Data processing systems100 may provide computer implemented services to users of the data processing system and/or devices operably connected to the data processing system. To do so, data processing system 100 may include out-of-band components (e.g., a network module, a management controller, etc.), and functionality that may allow data exchange between the out-of-band components independently from in-band components of data processing system 100. For more information regarding out-of-band components of data processing system 100, refer to discussion of FIG. 1B.

For example, out-of-band components of data processing systems 100 may (i) identify an occurrence of an emergency alert event (e.g., for data processing system 100 and/or a user of data processing system 100), (ii) initiate, in cooperation with management system 104, an emergency alert process, (iii) provide (iv) performing an action set to update the operation of the data processing system based on the identified policies, and/or (v) perform any other processes to facilitate computer implemented services.

Auxiliary device 102 may include any number and/or type of auxiliary devices (e.g., data processing systems, personal computing devices, monitoring devices, etc.) that may provide computer implemented services. To provide the computer implemented services, auxiliary device 102 may provide any type of data regarding state of health and/or activities of a user (operating the data processing system) to the hardware resources of data processing system 100 (e.g., and/or software components of the hardware resources). For example, auxiliary device 102 may be a glucose monitor worn by a user of the data processing system and may provide glucose readings of the user (e.g., during a period of time) to a glucose-related application hosted by the hardware resources of data processing system.

Management system 104 may include any number and/or type of management systems (e.g., data processing systems, servers, storage devices, user devices, etc.) that may provide computer implemented services, such as management services. To perform its functionality, management system 104 may communicate (e.g., exchange data) with the out-of-band components of data processing system 100 using out-of-band communication channels. For example, management system 104 may provide management services for data processing system 100 via a management controller of data processing system 100 (e.g., bypassing any in-band components of data processing system 100).

Management system 104 may cooperatively work with the management controller of a data processing system (e.g., 100) to perform management services. For example, management system 104 may exchange data and/or information relating to policies corresponding to the identified emergency alert events of the data processing system impacted. The policies may indicate various action sets to be performed by data processing systems in order to update operation of the data processing systems to mitigate potential impacts on the operation of the data processing systems due to the emergency alert events.

Communication system 108 may include two different and independent communication systems and various components of the system may be connected to one of the two independent communications systems. For example, data processing system 100 may be connected to a network communication system and utilize the network communication system to communicate with management system 104. In addition, emergency services system 106 may be connected to a telecommunications network and utilize the telecommunications network to communicate with management system 104.

In order to facilitate communications between data processing system 100 and emergency services system, management system 104 may establish a bridge between the two different communication systems.

Thus, the operation of data processing system 100 may be managed using out-of-band methods (e.g., using out-of-band components and via out-of-band communication channels). By doing so, the emergency alert events may be more likely to be identified, malicious activity and/or operating malfunctions may be more likely to be managed in a timely manner, and/or data processing system 100 may be more likely to provide the desired computer implemented services.

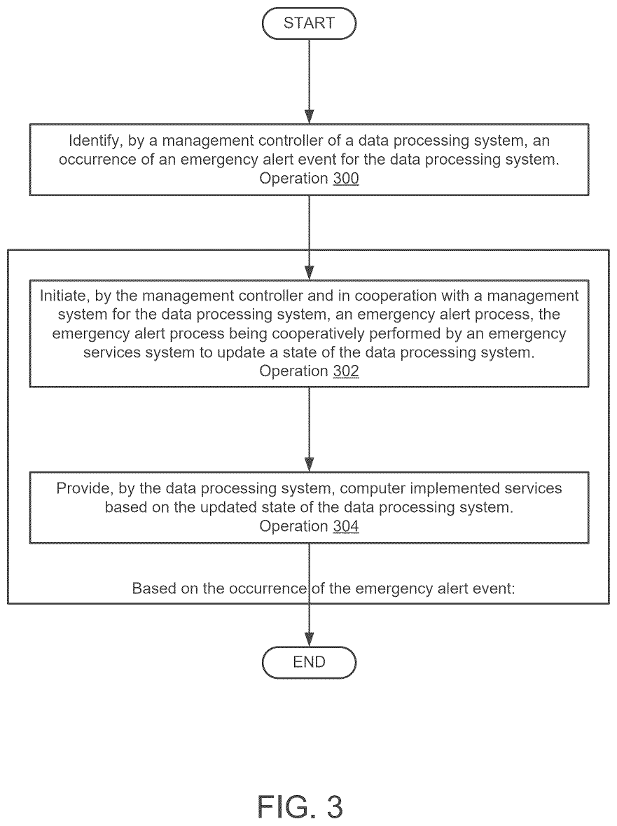

When providing their functionality, any of data processing system 100, auxiliary device 102, management system 104, and/or emergency service system 106 may perform all, or a portion of the methods shown in FIG. 3.

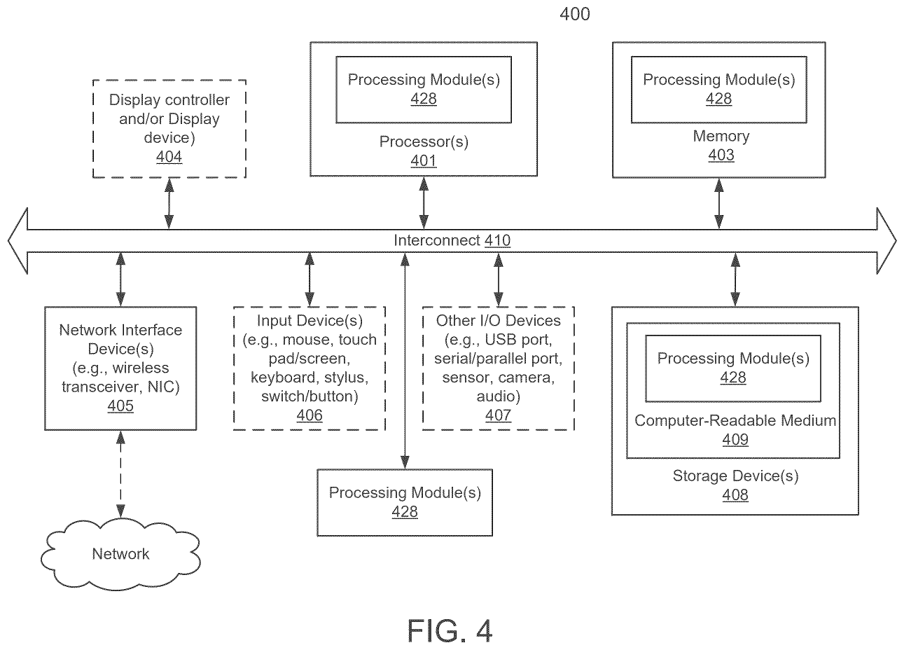

Any of (and/or components thereof) data processing system 100, auxiliary device 102, management system 104, and/or emergency service system 106 may be implemented using a computing device (also referred to as a data processing system) such as a host or a server, a personal computer (e.g., desktops, laptops, and tablets), a “thin” client, a personal digital assistant (PDA), a Web enabled appliance, a mobile phone (e.g., smartphone), an embedded system, local controllers, an edge node, and/or any other type of data processing device or system. For additional details regarding computing devices, refer to the discussion of FIG. 4.

In an embodiment, one or more of data processing system 100, auxiliary device 102, management system 104, and/or emergency service system 106 are implemented using an internet of things (IoT) device, which may include a computing device. The IoT device may operate in accordance with a communication model and/or management model known to any of data processing system 100, auxiliary device 102, management system 104, and/or emergency service system 106, and/or other devices.

Any of the components illustrated in FIG. 1A may be operably connected to each other (and/or components not illustrated) with communication system 108. In an embodiment, communication system 108 includes one or more networks that facilitate communication between any number of components. The networks may include wired networks and/or wireless networks (e.g., and/or the Internet). The networks may operate in accordance with any number and/or types of communication protocols (e.g., such as the internet protocol). Communication system 108 may include any number of in-band communication channel and/or out-of-band communication channels.

While illustrated in FIG. 1A as including a limited number of specific components, a system in accordance with an embodiment may include fewer, additional, and/or different components than those illustrated therein. For example, while the system of FIG. 1A shows a single management system (e.g., 104), it will be appreciated that the system may include any number of management systems.

Turning to FIG. 1B, a diagram illustrating data processing system 100 in accordance with an embodiment is shown. Data processing system 100 may be similar to any of data processing systems 100 shown in FIG. 1A.

To provide computer-implemented services, data processing system 100 may include any quantity of hardware resources 150. Hardware resources 150 may be in-band hardware components, and may include a processor operably coupled to memory, storage, and/or other hardware components. Hardware resources 150 may (e.g., via the processor) provide the computer-implemented services desired by users of data processing system 100.

The processor may host various management entities such as operating systems, drivers, network stacks, and/or other software entities that provide various management functionalities. For example, the operating system and drivers may provide abstracted access to various hardware resources.

To facilitate communication, hardware resources 150 may host a network stack that may facilitate packaging, transmission, routing, and/or other functions with respect to exchanging data with other devices. For example, the network stack may support transmission control protocol/internet protocol communication (TCP/IP) (e.g., the Internet protocol suite) thereby allowing the hardware resources 150 to communicate with other devices via packet switched networks and/or other types of communication networks.

The processor may also host various applications that provide the computer-implemented services. The applications may utilize various services provided by the management entities and use (at least indirectly) the network stack to communicate with other entities.

However, use of the network stack and the services provided by the management entities may place the applications at risk of indirect compromise. For example, if any of these entities trusted by the applications are compromised, these entities may subsequently compromise the operation of the applications. Additionally, if various drivers and/or the communication stack are compromised, communications to/from other devices may be compromised. If the applications trust these communications, then the applications may also be compromised.

For example, to communicate with other entities, an application may generate and send communications to a network stack and/or driver, which may subsequently transmit a packaged form of the communication via channel 170 to a communication component, which may then send the packaged communication (in a yet further packaged form, in some embodiments, with various layers of encapsulation being added depending on the network environment outside of data processing system 100) to another device via any number of intermediate networks (e.g., via wired/wireless channels 176 that are part of the networks).

To reduce the likelihood of the applications and/or other in-band entities from being indirectly compromised, data processing system 100 may include management controller 152 and network module 160. Each of these components of data processing system 100 is discussed below.

Management controller 152 may operate independently from hardware resources 150 and, therefore, hardware resources 150 may not host and/or manage operation of management controller 152. In addition, management controller 152 may be distinct from hardware resources 150 and, therefore, may be physically separate from hardware resources 150. Management controller 152 may also be operably connected to communication components of data processing system 100 via separate channels (e.g., 172) from the in-band components.

Management controller 152 may be implemented, for example, using a system on a chip or other type of independently operating computing device (e.g., independent from the in-band components, such as hardware resources 150, of a host data processing system 100).

Management controller 152 may provide various management functionalities for data processing system 100. For example, management controller 152 may monitor various ongoing processes performed by the in-band component, may manage power distribution, thermal management, and/or other functions of data processing system 100.

To do so, management controller 152 may be operably connected to various components via side band channels 174 (in FIG. 1B, a limited number of side band channels are included for illustrative purposes, it will be appreciated that management controller 152 may communication with other components via any number of side band channels). The side band channels may be implemented using separate physical channels, and/or with a logical channel overlay over existing physical channels (e.g., logical division of in-band channels). The side band channels may allow management controller 152 to interface with other components and implement various management functionalities such as, for example, general data retrieval (e.g., to snoop ongoing processes), telemetry data retrieval (e.g., to identify a health condition/other state of another component), function activation (e.g., sending instructions that cause the receiving component to perform various actions such as displaying data, adding data to memory, causing various processes to be performed), and/or other types of management functionalities.

For example, to reduce the likelihood of indirect compromise of an application hosted by hardware resources 150, management controller 152 may enable information from other devices to be provided to the application without traversing the network stack and/or management entities of hardware resources 150. To do so, the other devices may direct communications including the information to management controller 152. Management controller 152 may then, for example, send the information via side band channels 174 to hardware resources 150 (e.g., to store it in a memory location accessible by the application, such as a shared memory location, a mailbox architecture, or other type of memory-based communication system) to provide it to the application. Thus, the application may receive and act on the information without the information passing through potentially compromised entities. Consequently, the information may be less likely to also be compromised, thereby reducing the possibility of the application becoming indirectly compromised. Similar processes may be used to facilitate outbound communications from the applications.

Information provided to the application by management controller 152 may include, for example, instructions for implementation of computer-implemented services desired by users of data processing system 100.

To facilitate communication with other devices, data processing system 100 may include network module 160. Network module 160 may provide communication services for in-band components and out-of-band components (e.g., management controller 152) of data processing system 100.

To provide the above-described functionalities, network module 160 may include traffic manager 162, interfaces 164, and may host an instance of a TCP/IP stack to facilitate communication with other devices independently of any of the in-band components (e.g., does not rely on any hosted software, hardware components, etc.). Accordingly, compromise of any of hardware resources 150 and hosted component may not result in indirect compromise of network module 160, management controller 152, and entities hosted by management controller 152.

Management controller 152 may be operably connected to communication components of data processing system 100 via separate channels (e.g., 172) from the in-band components, and may implement or otherwise utilize a distinct and independent network stack (e.g., TCP/IP).

Consequently, management controller 152 may communicate with other devices independently of any of the in-band components (e.g., does not rely on any hosted software, hardware components, etc.). Accordingly, compromise of any of hardware resources 150 and hosted component may not result in indirect compromise of any management controller 152, and entities hosted by management controller 152.

Traffic manager 162 may include functionality to (i) discriminate traffic directed to various network endpoints advertised by data processing system 100, and (ii) forward the traffic to/from the entities associated with the different network endpoints. For example, to facilitate communications with other devices, network module 160 may advertise different network endpoints (e.g., different media access control address/internet protocol addresses) for the in-band components and out-of-band components. Thus, other entities may address communications to these different network endpoints. When such communications are received by network module 160, traffic manager 162 may discriminate and direct the communications accordingly (e.g., over channel 170 or channel 172, in the example shown in FIG. 1B, it will be appreciated that network module 160 may discriminate traffic directed to any number of data units and direct it accordingly over any number of channels).

Accordingly, traffic directed to management controller 152 may never flow through any of the in-band components. Likewise, outbound traffic from the out-of-band component may never flow through the in-band components.

To support inbound and outbound traffic, network module 160 may include any number of interfaces 164. Interfaces 164 may be implemented using any number and type of communication devices which may each provide wired and/or wireless communication functionality. For example, interfaces 164 may include a wide area network card, a WiFi card, a wireless local area network card, a wired local area network card, an optical communication card, and/or other types of communication components. These components may support any number of wired/wireless channels 176.

Thus, from the perspective of an external device, the in-band components and out-of-band components of data processing system 100 may appear to be two independent network entities, that may independently addressable, and otherwise unrelated to one another.

Network module 160 may utilize the instance of the TCP/IP stack to allow hardware resources 150 and/or management controller 152 to communicate with other devices via packet switched networks and/or other types of communication networks.

To facilitate management of data processing system 100 over time, hardware resources 150, management controller 152 and/or network module 160 may be positioned in separately controllable power domains. By being positioned in these separately controllable power domains, different subsets of these components may remain powered while other subsets are unpowered.

For example, management controller 152 and network module 160 may remain powered while hardware resources 150 is unpowered. Consequently, management controller 152 may remain able to communication with other devices even while hardware resources 150 are inactive. Similarly, management controller 152 may perform various actions while hardware resources 150 are not powered and/or are otherwise inoperable, unable to cooperatively perform various process, are compromised, and/or are unavailable for other reasons.

To implement the separate power domains, data processing system 100 may include a power source (e.g., 180) that separately supplies power to power rails (e.g., 184, 186) that power the respective power domains. Power from the power source (e.g., a power supply, battery, etc.) may be selectively provided to the separate power rails to selectively power the different power domains. A power manager (e.g., 182) may manage power from power source 180 that is supplied to the power rails (e.g., by providing instructions via side band channels 174). Management controller 152 may cooperate with power manager 182 to manage supply of power to these power domains. Management controller 152 may communicate with power manager 182 via side band channels 174 and/or via other means.

In FIG. 1B, an example implementation of separate power domains using power rails 184-186 is shown. The power rails may be implemented using, for example, bus bars or other types of transmission elements capable of distributing electrical power. While not shown, it will be appreciated that the power domains may include various power management components (e.g., fuses, switches, etc.) to facilitate selective distribution of power within the power domains.

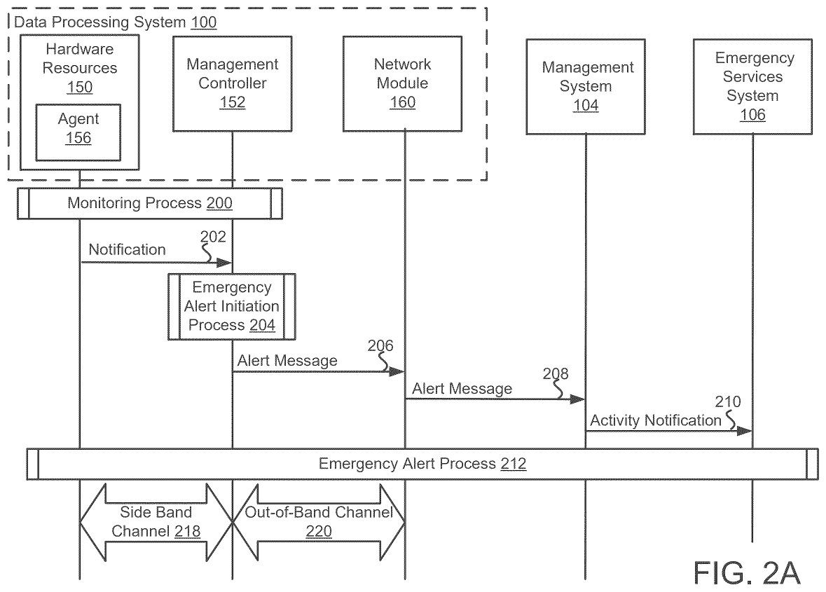

When providing its functionality, management controller 152 may perform all, or a portion, of the methods and operations described in FIG. 2A.

While illustrated in FIG. 1B with a limited number of specific components, a system may include additional, fewer, and/or different components without departing from embodiments disclosed herein.

To further clarify embodiments disclosed herein, interaction diagrams in accordance with an embodiment is shown in FIG. 2A. The interaction diagrams may illustrate examples of how data may be obtained and used within the systems of FIGS. 1A-1B.

In the interaction diagram, processes performed by and interactions between components of a system in accordance with an embodiment are shown. In the diagrams, components of the system are illustrated using a first set of shapes (e.g., 150, 152, etc.), located towards the top of each figure. Lines descend from these shapes. Processes performed by the components of the system are illustrated using a second set of shapes (e.g., 200, 208) superimposed over these lines.

Interactions (e.g., communication, data transmissions, etc.) between the components of the system are illustrated using a third set of shapes (e.g., 204, 206, etc.) that extend between the lines. The third set of shapes may include lines terminating in one or two arrows. Lines terminating in a single arrow may indicate that one-way interactions (e.g., data transmission from a first component to a second component) occur, while lines terminating in two arrows may indicate that multi-way interactions (e.g., data transmission between two components) occur.

Generally, the processes and interactions are temporally ordered in an example order, with time increasing from the top to the bottom of each page. For example, the interaction labeled as 204 may occur prior to the interaction labeled as 206. However, it will be appreciated that the processes and interactions may be performed in different orders, any may be omitted, and other processes or interactions may be performed without departing from embodiments disclosed herein.

The processes shown in FIG. 2A may be performed by any entity shown in the systems of FIGS. 1A-1B (e.g., a device similar to one of data processing system 100, management system 104, etc.) and/or another entity without departing from embodiments disclosed herein.

Turning to FIG. 2A, a first interaction diagram in accordance with an embodiment is shown. The first interaction diagram may illustrate processes and interactions that may occur during performance of an emergency service process to establish communication between a data processing system and an emergency service system.

To perform the emergency service process, monitoring process 200 may be performed. During monitoring process 200, management controller 152 may perform any type of monitoring processes on operation of the hardware resources (e.g., 150) hosted by data processing system (e.g., 100) to identify occurrences of an emergency alert event. The operation of hardware resources 150 may be indicative of occurrences of emergency alert events based on data received from auxiliary devices (e.g., auxiliary device 102). For example, auxiliary device 102 may provide data to one of the applications (e.g., corresponding to the auxiliary device and/or the type of data) hosted by hardware resources 150.

To monitor the operation of hardware resources 150, management controller 152 may program an agent (e.g., agent 156) hosted by the hardware resources to identify whether certain conditions have occurred. For example, agent 156 may be programed to screen for (e.g., identify) (i) a hokey combination being invoked (e.g., by a user of data processing system 100), (ii) information indicative of the occurrence of the emergency alert event being provided to the hardware resources (e.g., 150) by an auxiliary device (e.g., auxiliary device 102 shown in FIG. 1A), (iii) information indicative of the occurrence of the emergency alert event being provided to the hardware resources (e.g., 150) by a remote device, and/or (iv) any other indicators of an emergency alert event.

Auxiliary device may include a health state monitor, a personal computing device, and/or any other device with functionality to monitor (e.g., activity) of a user of the data processing system. For example, the health state monitor may include a heart rate monitor, a glucose monitor, and/or any other device monitoring a health condition of a user. The personal computing device may be, for example, a body worn computing device that includes (at least) one sensor for monitoring a user of the data processing system.

The remote device may be a safety system tasked with assisting in managing safety of users of the data processing system. For example, the safety system may be a fire management system and may provide information (e.g., alerts, messages, etc.) regarding indications of a fire that may negatively impact the safety of the users.

During monitoring process 200, agent 156 may identify that one of the conditions (e.g., associated with an emergency alert event) has been met and as such, agent 156 may generate a notification specifying the condition that may indicate an occurrence of an emergency alert event.

At interaction 202, the notification may be provided to management controller 152 by agent 156. For example, the notification may be provided to management controller 152 via (i) transmission via a message, (ii) storing in a storage with subsequent retrieval by management controller 152, (iii) via a publish-subscribe system where management controller 152 subscribed to updates from agent 156 thereby causing a copy of the notification to be propagated to management controller 152 and/or via other processes. The notification may be provided to management controller 152 via a sideband communication channel (e.g., side band channel 218). The sideband communication channel may be similar to side band channels 174 (e.g., previously described and shown in FIG. 1B).

Following receipt of the notification, management controller 152 may perform emergency alert initiation process 204. During emergency alert initiation process 204, management controller 152 may generate an alert message based on the notification and/or any other information regarding the state of the data processing system 100 (e.g., and/or state of the user of the data processing system).

At interaction 206, the alert message may be provided to network module 160 by management controller 152. For example, the alert message may be provided to network module 160 via an out-of-band communication channel (e.g., out-of-band channel 220). The out-of-band communication channel may be similar to the out-of-band channel (e.g., channel 172) previously described and shown in FIG. 1B.

At interaction 208, the alert message may be provided to management system 104 by network module 160. For example, the alert message may be provided to management system 104 using the network based communication system and via (i) transmission via a message, (ii) storing in a storage with subsequent retrieval by management system 104, (iii) via a publish-subscribe system where management system 104 subscribed to updates from network module 160 thereby causing a copy of the alert message to be propagated to management system 104 and/or via other processes.

Following receipt of the alert message, and at interaction 210, an activity notification may be provided to emergency services system 106 by management system 104. For example, the activity notification may be provided to emergency services system 106 using the telecommunications system and via (i) transmission via a message, (ii) storing in a storage with subsequent retrieval by emergency services system 106, (iii) via a publish-subscribe system where emergency services system 106 subscribed to updates from management system 104 thereby causing a copy of the activity notification to be propagated to emergency services system 106 and/or via other processes. By providing the activity notification to emergency services system 106, emergency services system 106 may receive a copy of the activity notification.

Once received, the activity notification may initiate performance of emergency alert process 212. During emergency alert process 212, emergency services system 106, management system 104, management controller 152, hardware resources 150, and/or any other components of the system may cooperatively perform any type of action sets to update the state of the data processing system (e.g., manage communication capabilities of the components).

For example, emergency services system 106 may initiate communication between a user of emergency services system 106 (e.g., dispatcher, operator, etc.) and hardware resources 150 of data processing system 100 by establishing a voice channel with the hardware resources (e.g., thereby updating the state of the data processing system) in order to facilitate the emergency services for the data processing system and/or a user of the data processing system.

As an additional example, emergency services system 106 may request telemetry data, geolocation data, and/or any other type of information from data processing system 100 in order to provide the applicable services to manage the type of emergency alert event.

Thus, as shown in the example of FIG. 2A, communication between data processing systems and remote entities may be established using out-of-band components of the data processing system. By doing so, the likelihood of being able to communicate with remote entities (e.g., emergency services system 106) that are required to cooperatively perform emergency services may be improved.

Any of the processes illustrated using the second set of shapes and interactions illustrated using the third set of shapes may be performed, in part or whole, by digital processors (e.g., central processors, processor cores, etc.) that execute corresponding instructions (e.g., computer code/software). Execution of the instructions may cause the digital processors to initiate performance of the processes. Any portions of the processes may be performed by the digital processors and/or other devices. For example, executing the instructions may cause the digital processors to perform actions that directly contribute to performance of the processes, and/or indirectly contribute to performance of the processes by causing (e.g., initiating) other hardware components to perform actions that directly contribute to the performance of the processes.

Any of the processes illustrated using the second set of shapes and interactions illustrated using the third set of shapes may be performed, in part or whole, by special purpose hardware components such as digital signal processors, application specific integrated circuits, programmable gate arrays, graphics processing units, data processing units, and/or other types of hardware components. These special purpose hardware components may include circuitry and/or semiconductor devices adapted to perform the processes. For example, any of the special purpose hardware components may be implemented using complementary metal-oxide semiconductor based devices (e.g., computer chips).

Any of the processes and interactions may be implemented using any type and number of data structures. The data structures may be implemented using, for example, tables, lists, linked lists, unstructured data, data bases, and/or other types of data structures. Additionally, while described as including particular information, it will be appreciated that any of the data structures may include additional, less, and/or different information from that described above. The informational content of any of the data structures may be divided across any number of data structures, may be integrated with other types of information, and/or may be stored in any location.

As discussed above, the components of FIGS. 1A-2A may perform various methods to improve secure operations of data processing systems by enforcing security posture-based policies via out-of-band communication methods. By enforcing the security posture-based policies, an out-of-band component of data processing systems may be able to implement the policies corresponding to the security posture of the data processing system in a timely manner and thereby, may decrease the likelihood of unauthorized access and/or operation of data processing systems.

FIG. 3 illustrates a method that may be performed by the components of the system of FIGS. 1A-2A. In the diagram discussed below and shown in FIG. 3, any of the operations may be repeated, performed in different orders, and/or performed in parallel with or in a partially overlapping in a timely manner with other operations. In the diagram, some of the operations (e.g., individually illustrated using a box) are surrounded by boxes with dashing lines to indicate, for example, components that are applicable to the boxes therein. The method described with respect to FIG. 3 may be performed by a data processing system and/or another device.

Turning to FIG. 3, a flow diagram illustrating a method of managing a data processing system in accordance with an embodiment is shown. The method may be performed, for example, by a data processing system, a management system, a communication system, a management controller, hardware resources, and/or other components illustrated in FIGS. 1A-2A.

At operation 300, an occurrence of an emergency alert event for a data processing system may be identified by a management controller of the data processing system. The occurrence of the emergency alert event may be identified by monitoring, by the management controller, operation of hardware resources. The operation of the hardware resources may be indicate of occurrences of the emergency alert event.

Monitoring the operation of the hardware resources may include programming, by the management controller, an agent hosted by the hardware resources. For example, the management controller may receive information usable to program the agent (e.g., hosted by the hardware resources) from an external entity (e.g., management system) and the management controller may provide instructions (including programming information) to the agent.

Upon receipt of the instructions and/or other information from the management controller, the agent may ingest the information and program to screen for at least one selected from one of the following, for example: invocation, by a user of the data processing system, a hotkey combination, providing, by an auxiliary device and to the hardware resources, information indicative of the occurrence of the emergency alert event, and/or information, by a remote device and the hardware resources, information indicative of the occurrence of the emergency alert event.

It will be appreciated that operations 302-304 may be performed based on the occurrence of the emergency alert event being identified during operation 300.

At operation 302, an emergency alert process may be initiated by the management controller and in cooperation with a management system for the data processing system. The emergency alert process may be cooperatively performed by an emergency services system to update a state of the data processing system. The emergency alert process may be initiated by (i) generating, by the management controller, a message indicating the occurrence of the emergency alert event, (ii) providing, by the management controller and using an out-of-band communication channel, the message to the management system, (iii) establishing, by the management system, a communication channel with the emergency services system and the management controller, and/or (iv) by any other methods.

At operation 304, computer implemented services may be provided by the data processing system based on the updated state of the data processing system. The computer implemented services may be provided by using the data processing system based on the updated state of the data processing system.

Using the methods illustrated in FIG. 3, embodiments disclosed herein may provide systems and methods usable to manage operations of data processing systems by identifying occurrences of emergency alert events that impact the data processing system and/or a user of the data processing system using out-of-band methods. By identifying the occurrence of emergency alert events, an emergency alert process may be initiated by a management controller of the data processing system and performing in cooperation with a management system and emergency services system in order to update a state of the data processing system. By updating the state of the data processing system, the likelihood of the desired computer implemented services being provided to a user of the data processing system may be improved.

Any of the components illustrated in FIGS. 1A-3 may be implemented with one or more computing devices. Turning to FIG. 4, a block diagram illustrating an example of a data processing system (e.g., a computing device) in accordance with an embodiment is shown. For example, system 400 may represent any of data processing systems described above performing any of the processes or methods described above. System 400 can include many different components. These components can be implemented as integrated circuits (ICs), portions thereof, discrete electronic devices, or other modules adapted to a circuit board such as a motherboard or add-in card of the computer system. Note also that system 400 is intended to show a high level view of many components of the computer system. However, it is to be understood that additional components may be present in certain implementations and furthermore, different arrangement of the components shown may occur in other implementations. System 400 may represent a desktop, a laptop, a tablet, a server, a mobile phone, a media player, a personal digital assistant (PDA), a personal communicator, a gaming device, a network router or hub, a wireless access point (AP) or repeater, a set-top box, or a combination thereof. Further, while only a single machine or system is illustrated, the term “machine” or “system” shall also be taken to include any collection of machines or systems that individually or jointly execute a set (or multiple sets) of instructions to perform any one or more of the methodologies discussed herein.

In one embodiment, system 400 includes processor 401, memory 403, and devices 405-407 via a bus or an interconnect 410. Processor 401 may represent a single processor or multiple processors with a single processor core or multiple processor cores included therein.

Processor 401 may represent one or more general-purpose processors such as a microprocessor, a central processing unit (CPU), or the like. More particularly, processor 401 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. Processor 401 may also be one or more special-purpose processors such as an application specific integrated circuit (ASIC), a cellular or baseband processor, a field programmable gate array (FPGA), a digital signal processor (DSP), a network processor, a graphics processor, a network processor, a communications processor, a cryptographic processor, a co-processor, an embedded processor, or any other type of logic capable of processing instructions.

Processor 401 may communicate with memory 403, which in one embodiment can be implemented via multiple memory devices to provide for a given amount of system memory. Memory 403 may include one or more volatile storage (or memory) devices such as random access memory (RAM), dynamic RAM (DRAM), synchronous DRAM (SDRAM), static RAM (SRAM), or other types of storage devices. Memory 403 may store information including sequences of instructions that are executed by processor 401, or any other device. For example, executable code and/or data of a variety of operating systems, device drivers, firmware (e.g., input output basic system or BIOS), and/or applications can be loaded in memory 403 and executed by processor 401. An operating system can be any kind of operating systems, such as, for example, Windows® operating system from Microsoft®, Mac OS®/iOS® from Apple, Android® from Google®, Linux®, Unix®, or other real-time or embedded operating systems such as VxWorks.

System 400 may further include IO devices such as devices (e.g., 405, 406, 407, 408) including network interface device(s) 405, optional input device(s) 406, and other optional IO device(s) 407. Network interface device(s) 405 may include a wireless transceiver and/or a network interface card (NIC). The wireless transceiver may be a WiFi transceiver, an infrared transceiver, a Bluetooth transceiver, a WiMax transceiver, a wireless cellular telephony transceiver, a satellite transceiver (e.g., a global positioning system (GPS) transceiver), or other radio frequency (RF) transceivers, or a combination thereof. The NIC may be an Ethernet card.

Input device(s) 406 may include a mouse, a touch pad, a touch sensitive screen (which may be integrated with a display device of optional graphics subsystem 404), a pointer device such as a stylus, and/or a keyboard (e.g., physical keyboard or a virtual keyboard displayed as part of a touch sensitive screen). For example, input device(s) 406 may include a touch screen controller coupled to a touch screen. The touch screen and touch screen controller can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with the touch screen.

IO devices 407 may include an audio device. An audio device may include a speaker and/or a microphone to facilitate voice-enabled functions, such as voice recognition, voice replication, digital recording, and/or telephony functions. Other IO devices 407 may further include universal serial bus (USB) port(s), parallel port(s), serial port(s), a printer, a network interface, a bus bridge (e.g., a PCI-PCI bridge), sensor(s) (e.g., a motion sensor such as an accelerometer, gyroscope, a magnetometer, a light sensor, compass, a proximity sensor, etc.), or a combination thereof. IO device(s) 407 may further include an imaging processing subsystem (e.g., a camera), which may include an optical sensor, such as a charged coupled device (CCD) or a complementary metal-oxide semiconductor (CMOS) optical sensor, utilized to facilitate camera functions, such as recording photographs and video clips. Certain sensors may be coupled to interconnect 410 via a sensor hub (not shown), while other devices such as a keyboard or thermal sensor may be controlled by an embedded controller (not shown), dependent upon the specific configuration or design of system 400.

To provide for persistent storage of information such as data, applications, one or more operating systems and so forth, a mass storage (not shown) may also couple to processor 401. In various embodiments, to enable a thinner and lighter system design as well as to improve system responsiveness, this mass storage may be implemented via a solid state device (SSD).

However, in other embodiments, the mass storage may primarily be implemented using a hard disk drive (HDD) with a smaller amount of SSD storage to act as a SSD cache to enable non-volatile storage of context state and other such information during power down events so that a fast power up can occur on re-initiation of system activities. Also a flash device may be coupled to processor 401, e.g., via a serial peripheral interface (SPI). This flash device may provide for non-volatile storage of system software, including a basic input/output software (BIOS) as well as other firmware of the system.

Storage device 408 may include computer-readable storage medium 409 (also known as a machine-readable storage medium or a computer-readable medium) on which is stored one or more sets of instructions or software (e.g., processing module, unit, and/or processing module/unit/logic 428) embodying any one or more of the methodologies or functions described herein. Processing module/unit/logic 428 may represent any of the components described above. Processing module/unit/logic 428 may also reside, completely or at least partially, within memory 403 and/or within processor 401 during execution thereof by system 400, memory 403 and processor 401 also constituting machine-accessible storage media. Processing module/unit/logic 428 may further be transmitted or received over a network via network interface device(s) 405.

Computer-readable storage medium 409 may also be used to store some software functionalities described above persistently. While computer-readable storage medium 409 is shown in an exemplary embodiment to be a single medium, the term “computer-readable storage medium” should be taken to include a single medium or multiple media (e.g., a centralized or distributed database, and/or associated caches and servers) that store the one or more sets of instructions. The terms “computer-readable storage medium” shall also be taken to include any medium that is capable of storing or encoding a set of instructions for execution by the machine and that cause the machine to perform any one or more of the methodologies of embodiments disclosed herein. The term “computer-readable storage medium” shall accordingly be taken to include, but not be limited to, solid-state memories, and optical and magnetic media, or any other non-transitory machine-readable medium.

Processing module/unit/logic 428, components and other features described herein can be implemented as discrete hardware components or integrated in the functionality of hardware components such as ASICS, FPGAs, DSPs or similar devices. In addition, processing module/unit/logic 428 can be implemented as firmware or functional circuitry within hardware devices. Further, processing module/unit/logic 428 can be implemented in any combination hardware devices and software components.

Note that while system 400 is illustrated with various components of a data processing system, it is not intended to represent any particular architecture or manner of interconnecting the components; as such details are not germane to embodiments disclosed herein. It will also be appreciated that network computers, handheld computers, mobile phones, servers, and/or other data processing systems which have fewer components or perhaps more components may also be used with embodiments disclosed herein.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Embodiments disclosed herein also relate to an apparatus for performing the operations herein. Such a computer program is stored in a non-transitory computer readable medium. A non-transitory machine-readable medium includes any mechanism for storing information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices).

The processes or methods depicted in the preceding figures may be performed by processing logic that comprises hardware (e.g. circuitry, dedicated logic, etc.), software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

Embodiments disclosed herein are not described with reference to any particular programming language. It will be appreciated that a variety of programming languages may be used to implement the teachings of embodiments disclosed herein.

In the foregoing specification, embodiments have been described with reference to specific exemplary embodiments thereof. It will be evident that various modifications may be made thereto without departing from the broader spirit and scope of the embodiments disclosed herein as set forth in the following claims. The specification and drawings are, accordingly, to be regarded in an illustrative sense rather than a restrictive sense.

What is claimed is:

1. A method for managing operation of a data processing system, the method comprising:
identifying, by a management controller of the data processing system, an occurrence of an emergency alert event for the data processing system;
based on the occurrence of the emergency alert event:
initiating, by the management controller and in cooperation with a management system for the data processing system, an emergency alert process, the emergency alert process being cooperatively performed by an emergency services system and at least hardware resources of the data processing system to update a state of the data processing system; and
providing, by the data processing system, computer implemented services based on the updated state of the data processing system.

2. The method of claim 1, wherein identifying the occurrence of the emergency alert event comprises:
monitoring, by the management controller, operation of the hardware resources, the operation of the hardware resources being indicative of occurrences of the emergency alert event.

3. The method of claim 2, wherein monitoring the operation of the hardware resources comprises:
programming, by the management controller, an agent hosted by the hardware resources.

4. The method of claim 3, wherein the agent is programmed to screen for at least one selected from a group consisting of:
invocation, by a user of the data processing system, a hotkey combination;
providing, by an auxiliary device and to the hardware resources, information indicative of the occurrence of the emergency alert event; and
information, by a remote device and the hardware resources, information indicative of the occurrence of the emergency alert event.

5. The method of claim 4, wherein the auxiliary device is one selected from a group consisting of:
a health state monitor; and
a personal computing device.

6. The method of claim 5, wherein the health state monitor is one selected from a group consisting of:
a heart rate monitor; and
a glucose monitor.

7. The method of claim 5, wherein the personal computing device is a body worn computing device that comprises at least one sensor for monitoring a user of the data processing system.

8. The method of claim 4, wherein the remote device is a safety system tasked with assisting in managing safety of users of the data processing system.

9. The method of claim 7, wherein the safety system is a fire management system.

10. The method of claim 1, wherein the data processing system comprises a network module adapted to separately advertise network endpoints for the management controller and the hardware resources of the data processing system, the network endpoints being usable by the management system and the emergency services system to address communications to the hardware resources using an in-band communication channel and the management controller using the out-of-band communication channel.

11. The method of claim 10, wherein the management controller and the network module are on separate power domains from the hardware resources so that the management controller and the network module are operable while the hardware resources are inoperable.

12. The method of claim 10, wherein the out-of-band communication channel runs through the network module, and the in-band communication channel that services the hardware resources also runs through the network module.

13. The method of claim 10, wherein the network module hosts a transmission control protocol/internet protocol (TCP/IP) stack to facilitate network communications via the out-of-band communication channel.

14. A non-transitory machine-readable medium having instructions stored therein, which when executed by a processor, cause the processor to perform operations for managing operation of a data processing system, the operations comprising:
identifying, by a management controller of the data processing system, an occurrence of an emergency alert event for the data processing system;
based on the occurrence of the emergency alert event:
initiating, by the management controller and in cooperation with a management system for the data processing system, an emergency alert process, the emergency alert process being cooperatively performed by an emergency services system and at least hardware resources of the data processing system to update a state of the data processing system; and
providing, by the data processing system, computer implemented services based on the updated state of the data processing system.

15. The non-transitory machine-readable medium of claim 14, wherein identifying the occurrence of the emergency alert event comprises:
monitoring, by the management controller, operation of the hardware resources, the operation of the hardware resources being indicative of occurrences of the emergency alert event.

16. The non-transitory machine-readable medium of claim 15, wherein monitoring the operation of the hardware resources comprises:
programming, by the management controller, an agent hosted by the hardware resources.

17. The non-transitory machine-readable medium of claim 16, wherein the agent is programmed to screen for at least one selected from a group consisting of:
invocation, by a user of the data processing system, a hotkey combination;
providing, by an auxiliary device and to the hardware resources, information indicative of the occurrence of the emergency alert event; and
information, by a remote device and the hardware resources, information indicative of the occurrence of the emergency alert event.

18. A data processing system, comprising:
a processor; and
a memory coupled to the processor to store instructions, which when executed by the processor, cause the processor to perform operations for managing operation of the data processing system, the operations comprising:
identifying, by a management controller of the data processing system, an occurrence of an emergency alert event for the data processing system;
based on the occurrence of the emergency alert event:
initiating, by the management controller and in cooperation with a management system for the data processing system, an emergency alert process, the emergency alert process being cooperatively performed by an emergency services system and at least hardware resources of the data processing system to update a state of the data processing system; and
providing, by the data processing system, computer implemented services based on the updated state of the data processing system.

19. The data processing system of claim 18, wherein identifying the occurrence of the emergency alert event comprises:
monitoring, by the management controller, operation of the hardware resources, the operation of the hardware resources being indicative of occurrences of the emergency alert event.

20. The data processing system of claim 19, wherein monitoring the operation of the hardware resources comprises:
programming, by the management controller, an agent hosted by the hardware resources.
